(19) United States
(12) Patent Application Publication
(10) Pub. No.: US 2008/0254471 A1
(43) Pub. Date: Oct. 16, 2008

(54) Apparatus and Method for Microbial and Forensic Sampling and Manipulation

(75) Inventor: William Nicholas Bordano, Fair Oaks Ranch, TX (US)

Correspondence Address: WONG, CABELLO, LUTSCH, RUTHERFORD & BRUCCULERI,;L.L.P., 20333 SH 249, SUITE 600, HOUSTON, TX, 77070, US

(73) Assignee: ALAMO SCIENTIFIC, San Antonio, TX (US)

(21) Appl. No.: 12/089,971

(22) PCT Filed: Dec. 28, 2005
(86) PCT No.: PCT/US2005/047295

§ 371 Date: Apr. 11, 2008

Related U.S. Application Data
(60) Provisional application No. 60/726,739, filed on Oct. 13, 2005.

(51) Int. Cl. C12Q 1/68; A61B 10/02; C12Q 1/24

(52) U.S. Cl. 435/6; 600/562; 600/563; 435/30

(57) ABSTRACT

The present invention provides a sterile collection package and methods for sample procurement and manipulation including microbial and forensic testing. The collection package comprises a tissue or sample collection container with a sealable opening, at least one drain container, and at least one tubing set that connects the tissue or sample collection container and the drain container, the tubing set further optionally comprising a sampling hub for withdrawing sample fluid for microbial and forensic testing. Samples, such as donated tissues, diseased tissues and article and tissues for forensic analysis are mixed with a sampling solution in the collection container and aliquots of the sampling solution are withdrawn through the sampling hub for microbial and/or forensic testing.

### CROSS REFERENCE TO RELATED APPLICATIONS

This application claims priority based on U.S. Provisional Application Ser. No. 60/726,739 filed Oct. 13, 2005.

### FIELD OF THE INVENTION

The present disclosure provides methods and apparatus for sterile manipulation of tissues and samples for microbial and forensic testing including contagion sampling on tissues or organs that may be used for transplantation, as well as testing on diseased tissue and surgical instrumentalities.

### BACKGROUND OF THE INVENTION

With regard to human tissue transplantation, once permission is granted from a family member of the tissue donor, personnel of tissue center will begin “procurement” which is essentially a sterile surgical procedure where the bones, tendons, and any other viable tissues are taken out of the tissue donor to be further processed. Under regulations of the FDA and other governing bodies around the world, these body tissues must be tested for certain contagious agents. If certain organisms are found, the donated tissues cannot be used for transplantation.

Currently, the donated tissues are screened for microbial contamination by the following procedure. After the tissues are harvested from the donor, members of the procurement team would collect microbial testing samples by rubbing sterile swab sticks on the harvested tissue pieces. These sterile swabs are then shipped back to the tissue center for microbial culture. Collecting microbial testing samples in this manner is a very labor intensive process. Microbial organisms may not be detected because they die on the swab during transport, or the microbes are missed because they are located in areas not touched by the sterile swabs or because the sample size is not sufficient to detect a scarce or low titer pathogen. Contagious agents such as viruses and, particularly atypical agents such as prion agents are likely to be undetectable by limited sampling. In particular, a number of cases of transmissible spongiform encephalopathies (TSEs) caused by prion agents have been the result of transplanted tissues. As a consequence, recipients of dura mater and cornea transplants are regarded by the World Health Organization as “at risk for TSE.” Although the highest infectivity tissues for TSE are tissues of the central nervous system (CNS), infectivity has also been found with organs outside of the CNS including lung, liver, kidney, spleen/lymph nodes and placenta.

Also needed are apparatus and methods for comprehensive sampling of surgical instrumentalities and forensic samples that provides for collection, sampling and storage without further contamination of the samples. In view of the above remarks, the present invention discloses methods and apparatus that will provide better and more efficient methods of manipulating and screening samples for contagious agents and organisms as well as other foreign contaminants.

### SUMMARY OF THE DISCLOSURE

The present invention provides methods and apparatus for collecting samples for microbial and other foreign contaminant testing into a collection package that enables manipulation of the samples in a sterile environment, wherein the manipulation is one or more of washing of the sample or article, sampling for contamination and, storage and/or transport of the sample.

In one embodiment, a collection package is provided herein for tissue procurement and microbial testing. Alternatively, the collection package can be employed for washing and/or storage of collected tissues even if microbial testing is not required. In one embodiment of the invention, tissues for collection include bone, ligaments, musculoskeletal tissue, vascular tissue including venous and arterial tissue, skin, dura mater, heart valves, ocular tissue including corneas, vascularized organs for transplantation, and cells contained or associated with tissues and organs. The collection package provides for suffusion of the tissue in storage and/or transport solutions.

In one embodiment, a collection package is provided for microbial testing of surgical tools and devices. The collection package provides for transport, testing and storage of surgical tools that have been used in procedures that may post a risk of infection if reused after conventional sterilization. After use, the tools are placed in the package, sealed and removed from the surgical arena. After collection the tools are safely manipulated within the package, wherein the manipulation is one or more of washing of the instruments in the container, sampling of the wash solution for microbial contamination and storage of the instruments until test results are received. Test results determine the cleaning, sterilization or disposal protocol for the instruments.

In one embodiment of the invention, a collection package is provided for microbial testing of surgical specimens. The collection package provides for packaging and storage of the specimen and for comprehensive testing of the specimen for occult and/ or drug resistant organisms including bacteria and parasites.

In another embodiment of the invention, the collection package is used in forensic applications for collection, testing and storage of samples collected from crime victims and crime scenes. The washing and sampling capabilities of the present invention may be employed to provide large volume comprehensive sampling for forensic analysis, including microbial, DNA, locality specific contaminants, and toxicology screens.

In one embodiment, the collection package comprises a collection container dimensioned to accommodate a sample/tissue and a volume of washing or sampling solution. The collection container features a sealable opening so that the container can be sealed after the sample is deposited therein. The collection container is connected through at least one tubing set to at least one drain container that is dimensioned to accommodate at least a portion of the washing or sampling solution. The tubing set provides a controllable fluid path between the collection container and the drain container. The washing or sampling solution can be added to the collection container when the sample or tissue is added to the container. Alternatively the washing or sampling solution can be added to the collection container and/or drain container during manufacture of the collection package.

In one embodiment the washing or sampling solution is a simple physiologically compatible solution. In other embodiments, the washing or sampling solution is a more complex mixture that is particularly suitable for enhancing viability of organs for donation. Depending on the tissue, the washing or sampling solution, which doubles as a tissue preservation solution, is particularly suitable for freezing preservation of the tissues. In other embodiments, the system provides for sterile treatment of harvested tissue with a succession of solutions including, for example, two or more of washing, sampling and transport or storage solutions.

In general, the tubing set comprises at least one transfer hub disposed between a collection container and a drain container. Where used for microbial or forensic testing the transfer hub can be considered a sampling hub and portions of the sampling solution are transferable through the hub into sampling tubes/bottles for microbial or forensic testing. Withdrawal of fluid samples can be done in a number of ways generally practiced in the art. For example, the transfer hub can be a luer lock fitting so that a syringe can be attached to pull sample fluid out. Alternatively, the transfer hub can include a stopper though which a needle can be advanced for removal of fluid.

In one embodiment, the hub can be a needle hub that is provided with a needle when it is manufactured. In another embodiment, the needle hub can include a bell that provides for a sterile environment for advancement of the needle into one or more sampling tubes/bottles. The transfer hub may include a combination of one or more of a luer lock fitting, a needle penetrable stopper, a septum syringe port, a safety connection device, a valve, and a needle hub. For example, the transfer hub may comprise a valve having a luer lock fitting or a septum syringe port disposed on one end.

In one embodiment, tubing set includes a plurality of ports, each adapted for collecting the desired type of sample desired for contagion or other testing including a needle hub for withdrawal of fluid samples directly into culture tubes or bottles, such as for example anaerobic and aerobic blood culture tubes and/or bottles. In one embodiment a further hub is provided on the tubing for delivering a relatively large portion of sampling fluid to a filtration apparatus whereby a volume of fluid can be concentrated by filtration and the filter disk subsequently tested for microbes and other particulate matter. As used herein, the term “contagion” or “microbe” refers to any agent that is transmissible and results in disease including bacteria, mycoplasma, parasites, fungi, viruses, and atypical agents such as prion agents.

In one embodiment, an integrated procurement and sampling set is provided that includes culture tubes and/or bottles. The present invention also provides methods of using the collection package disclosed for sample/tissue procurement and microbial or forensic testing. In one embodiment, the biological specimens such as donated tissues or organs, diseased tissue, forensic samples, or surgical instrumentalities are first agitated with a sampling solution in the collection container. Part of the sampling solution is then drained via a sampling hub into one or more microbial culture tubes and/or culture bottles. The remaining sampling solution is drained into the drain container. Aliquots of sampling solution in the drain container can be further tested. Large volume collection and concentration for bacterial testing or forensics can be provided by draining the solution through a filter which is then tested in microbial culture or is used to collect and concentrate particulates. Any remainder fluid in the drain container can be stored for archival testing, storage or discarding.

The foregoing summary is not intended to summarize each potential embodiments or every aspect of the present disclosure.

### BRIEF DESCRIPTION OF THE DRAWINGS

The foregoing summary, preferred embodiments, and other aspects of subject matter of the present disclosure will be best understood with reference to a detailed description of specific embodiments, which follows, when read in conjunction with the accompanying drawings, in which:

FIG. 1 illustrates one embodiment of the collection package disclosed herein having a single transfer hub.

FIG. 2 illustrates one embodiment of the collection package disclosed herein having a plurality of transfer and or sampling hubs.

FIG. 3 illustrates one embodiment of the collection package disclosed herein including a clamp closure for the tissue container.

FIG. 4 illustrates one embodiment of the collection package disclosed including a stepwise depiction of insertion of a tissue into the tissue container and sealing of the container, including placement in a protective overbag or pouch.

The disclosed method and apparatus are susceptible to various modifications and alternative forms within the spirit of the invention. The figures and written description are not intended to limit the scope of the inventive concepts in any manner. Rather, the figures and written description are provided to illustrate the inventive concepts to a person skilled in the art by reference to particular embodiments, as required by 35 U.S.C. §112.

### DETAILED DESCRIPTION

The present invention provides methods and apparatus for sterile manipulation of samples, tissues and organs for microbial or forensic testing, and for storage and/or transportation of such samples. Where used for tissues for transplantation, an integral procurement, washing, sampling, and storage or transportation system is provided.

Disclosed herein is a collection package for tissue procurement and manipulation including, if desired, contagion testing, comprising a tissue container with a sealable access opening, one or more drain containers(s), and at least one sterile tubing set for providing a controllable fluid path between the tissue container and the drain container, wherein the tubing set comprises at least one transfer or sampling hub disposed between the tissue container and the drain container. In one embodiment of the invention, contagion testing of tissues for transplantation is provided by removal of washing or sampling fluid that has been thoroughly agitated with the tissues.

As used herein the term “tissues” includes human and other animal tissues. The term “tissues for transplantation” includes tissues that are intended for implantation, transplantation, or transfer into a human or other animal recipient. Examples include, but are not limited to: bone, ligaments, musculoskeletal tissue, vascular tissue including venous and arterial tissue, skin, dura mater, heart valves, ocular tissue including corneas, and vascularized organs for transplantation.

Tissues may also include diseased or unwanted tissues removed from a patient during surgical procedures including for example surgical reconstruction and surgical debridement of wounds. Where used in such indications, the collection package allows for comprehensive sampling of removed diseased or unwanted tissue for microbial testing, such as detection of occult and antibiotic resistant bacteria, and for parasites.

The tissue container and/or drain container can be made of rigid, semi-rigid, or flexible plastic materials generally available in the art. Well-known suppliers of flexible plastic containers and bags include, but are not limited to, Baxter International, Inc. (Deerfield, Ill.), Terumo Medical Corporation (Somerset, N.J.), and Amsino (Pamona, Calif.). Thus, the tissue container and drain container can be constructed from a number of materials that can be formed into containers of desirable dimensions, are sterilizable and are of pharmaceutical quality. Pharmaceutically compatible plastic materials presently available include, without limitation, polyvinyl chloride (PVC), polypropylene, polyethylene, polypropylene-ethylene copolymers, styrene-ethylene and styrene-butylene copolymers, and multilayer combinations thereof.

The tissue container and/or drain container, if constructed as a flexible container, such as in a bag form, can be formed by any method generally available in the art. For example, radio frequency (RF) sealers are widely used in the manufacture of medical devices including blood storage bags made with materials conducive to excitation by RF, including for example polyvinyl chloride.

Depending on the uses, the tissue container can be made into different sizes and configurations. For an example, the tissue container is optionally dimensioned in a variety of sizes depending on the size of the tissue to be deposited therein. For example, for small tissues or organs a relatively small tissue container such as a 4″×6″ or smaller container may be desirable. Intermediate sizes, such as for example 10″×9″ may be desirable for medium sized tissues and/or organs, while larger sizes for larger tissues such as complete adult organs are provided, such as for example, 26″×8″ or 34″×12″ containers. The volume of testing and/or storage solution that can be disposed in the tissue container at any one time will necessarily be dependent on the size of the tissue container as well as the volume of fluid that is required for removal for each desired test.

Preferably, the tissue container can maintain and store biological samples at freezing temperatures of −20° C. or lower. The tissue container also has an access opening that can be opened and closed so that tissue samples can be deposited into and removed from the container. In one embodiment, the tissue container and/or drain container, is dimensioned to form a stand-up pouch having a reclosable access opening on the top of the bag when the bag is sitting upright.

The access opening is dimensioned to accommodate deposition of the tissues without contamination or contusion and can be opened and closed by a releasable closure such as a rib and groove closure. Rib and groove closures include a projection that is integral with and extends along at least a portion of the opening and which interlocks with a mating recess extending along an opposite portion of the opening. Zip-lock type closures are examples of rib and groove closures as are slider zipper type closures.

Alternatively, the access opening may be closed using a suitable clamp-like closure may be formed of two essentially rigid members that extend substantially completely along the opposite sides of the container access opening and cooperate to press or hold the access opening shut. The members can be separate bars or opposed portions of a one piece clip.

In one embodiment, the access opening of the container can be welded or fused shut after the tissue has been placed in the container. For example, heat sealing thermal impulse sealers or constant heat bag sealers may be employed and are widely available, including those manufactured to be FDA compliant for medical indications. Other methods of heat sealing may be employed such as radio frequency (RF) sealing or ultrasonic sealing although technologies would need to be adapted to provide portability, ease of use and safety considerations to make them suitable for medical environments. Use of a clamp closure or a welded closure may be used in lieu of, or in addition to, a resealable closure such as a releasable adhesive or rib and groove closure.

Alternatively, the tissue container and/or drain container can be rigid or semi-rigid, in which case suitable closures would be utilized including for example, snap top closures, threaded closures, etc. without departing from the conceptual framework of an integrated collection set including tubing for microbial and/or forensic testing.

The collection container is connected to the drain container by at least one sterile tubing set that provides a controllable fluid path between the collection container and the drain container. Movement of fluid solutions, such as washing, sampling and/or storage fluid, between the collection container and the drain container is controllable by one or more clamps, slide closures and/or valves or other similar closure devices on the tubing. The tubing closures can be placed on the tubing set during manufacture or may be separate from the tubing set. For example, hemostats and/or plastic or nylon one piece hinged closures may be used to clamp the tubing wherever desired. In a preferred embodiments the tubing set includes clamps mounted on the tubing for convenience.

The tubing set further comprises at least one transfer hub disposed between the collection container and the drain container for addition or removal of fluid. In one embodiment, the transfer hub is a sampling hub for removal of fluid for microbial testing or other testing. As a conduit for fluid withdrawal, the sampling hub can be configured in a number of ways generally known in the art. For example, the transfer/sampling hub can comprise a luer lock fitting, a stopper, a septum syringe port and/or a needle hub comprising a needle.

The procurement and testing set can be provided if desired in a sterile or sterilizable peelable overpouch. Alternatively, the collection package can be wrapped, such as for example in cloth or paper, for sterilization and/or delivery to the operating suite as are other surgical instruments.

In general, representative examples of washing or sampling solutions for transplantation purposes include physiologically compatible solutions including simple solutions having small molecule (crystalloid) solutes including but not limited to phosphate buffers, physiological (0.9%) saline, Ringer's lactate, 5% dextrose in water, and/or simple physiological glucose solutions. Washing or sampling solutions can alternatively be more complex solutions that may include two or more constituents including electrolytes, osmotic agents, buffers, and chelating agents. Examples of electrolytes include, but are not limited to, physiologically compatible solutions of sodium, chloride, potassium, calcium, and magnesium ions.

The term osmotic agent as used herein refers agents that can be used to control the flow of water into and out of cells and ultimately tissues by virtue of their particle concentration in water. Typical agents used to control osmosis include but are not limited to glucose, mannitol, potassium and lactobionate. Oncotic agents may be considered a subset of osmotic agents that can be used to control the flow of water into and out of the intravascular compartment by virtue of having a size sufficient to resist ready transfer across capillary beds into the interstitial spaces of tissues. Oncotic pressure is thus sometimes referred to as colloidal osmotic pressure. Examples of oncotic agents include, but are not limited to, colloidal solutes including proteins such as albumin and high molecular weight hemoglobin, dextrans, hydroxyethyl starch, and other glucose polymers, and synthetic polymers such as polyvinyl pyrrolidone (PVP), polyethylene glycol (PEG), polyoxyethlene-polyoxypropylene copolymers (poloxamers). Certain of these agents also serve as cryoprotectants.

Examples of buffers include, but are not limited to, solutions containing one or more of: sodium bicarbonate (NaHCO₃); lactate, acetate, gluconate, citrate, and/or pyruvate carboxylic acids; 3-[N-tris(Hydroxy-methyl) ethylamino]-2-hydroxyethyl]-1-piperazinepropanesulfonic acid (EPPS); N-2-Hydroxyethylpiperazine-N′-2-hydroxypropanesulfonic acid (HEPES); 3-(N-Morpholino) propanesulfonic acid (MOPS); 2-([2-Hydroxy-1,1-bis(hydroxymethyl)ethyl]amino) ethanesulfonic acid (TES); Tris[hydroxymethyl]-aminomethane (THAM); and Tris[hydroxymethyl]methyl aminomethane (TRIS).

Alternatively, the washing or sampling solution can be adapted to a particular tissue or organ cold storage solution. Examples of such solutions include organ specific cold perfusion solutions such as “cardioplegia” for the heart and “pulmonoplegia” for the lungs, which typically contain various ratios of lactate, adenosine, potassium, sodium, bicarbonate, mannitol and a free radical scavenger. Examples of commercial solutions include ViaSpan® Cold Storage Solution (UW solution developed by the University of Wisconsin and available from DuPont Merck Pharma), Unisol® (Organ Recovery Systems, Inc. Chicago, Ill.), and Hypothermosol® (BioLife Solutions, Inc., Ewing, N.J.).

The collection container and/or the drain container can be pre-filled during manufacturing with a volume of fluid. For example, in one embodiment in which the collection and/or drain containers are flexible collapsible bags, a volume of fluid such as washing or sampling solution can be disposed in both the collection container and drain container during manufacturing to prevent the walls of the containers from sticking together. In one embodiment of the invention, the collection and/or drain containers are prefilled during manufacturing with a volume of a simple aqueous solution such as normal saline to which a concentrate of a desired organ preservation solution can be added. In such a way, each transplant center can utilize its preferred preservation solution while the advantage of prefilling with an aqueous solution can also be realized.

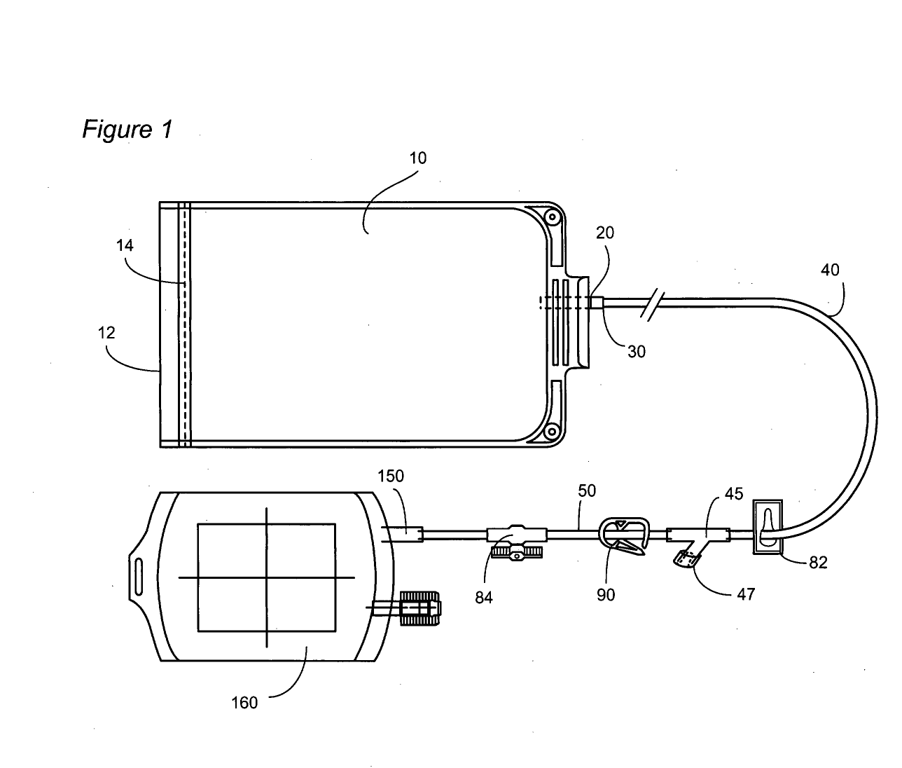

FIG. 1 depicts one embodiment of the collection package. A collection or tissue container 10 is connected through tube connector 20, tube sleeve 30 and tubing 40 to a T- or Y-connector 45. The open and closable end 12 of the collection or tissue container 10 can be closed by a self sealing rib and groove closure 14. Alternatively the open and closable end 12 can be sealed by a clamp or by fusion of the container material such as by heat. Connector 45 disposed on tubing 40 may function as a transfer hub by inclusion of one or more ports such as septum syringe port 47 for addition of fluid to the system or for harvesting aliquots of sampling fluid for microbial testing. Alternatively, the connector 45 can provide a luer lock or other means of attaching sample collection apparatus. Connector 45 also provides a fluid connection to tubing 50, which, in the depicted embodiment, is connected via tube sleeve 150 to a drain bag 160. In one embodiment, the drain container 160 can be a blood collection bag generally used in the art. The drain container is preferably dimensioned to accommodate any portions of the original volume of wash, sample or storage solution from the tissue/collection container that are not otherwise used for washing, testing or storage. The drain container can be either stored or disposed of a bio-hazardous waste as desired. Movement of the sampling fluid between the tissue/collection container and the drain container is controllable if desired by one or more devices such as clamps 90, slide closures 82, valves 84 and/or other similar closure devices that are mounted on the tubing during manufacture or, alternatively, clipped onto the tubing during procedures. Other suitable closures can be alternatively employed such as, for example, roller clamps.

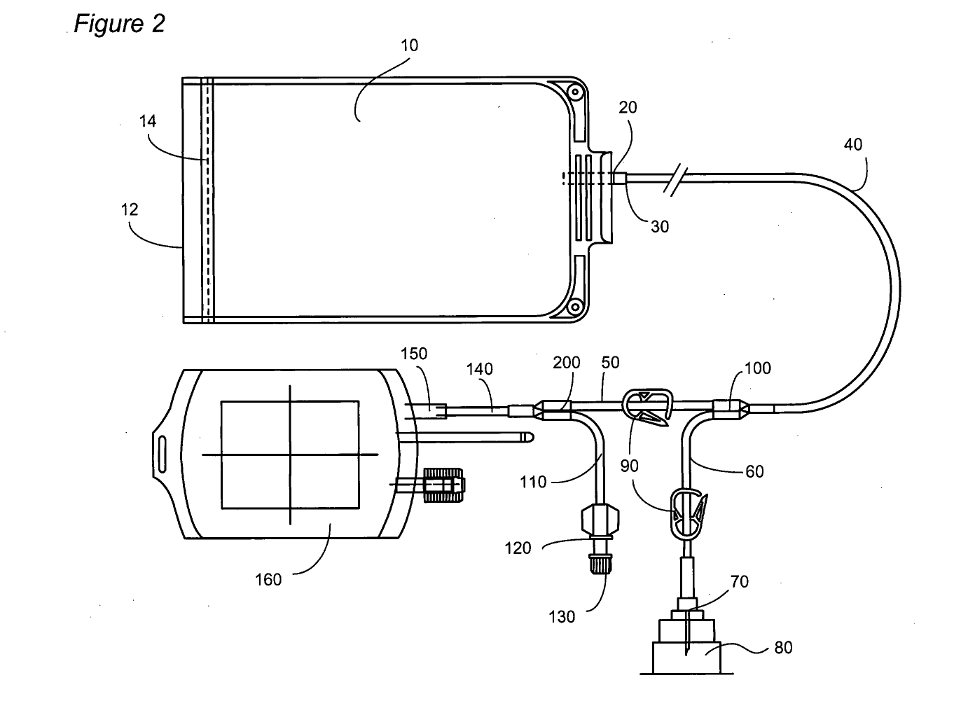

FIG. 2 depicts one embodiment of the collection package including a plurality of sampling ports. A tissue/collection container 10 is connected through tube connector 20, tube sleeve 30 and tubing 40 to a 3-way connector 100. The access opening or lip 12 of the tissue/collection container 10 is closed, such as for example, by a pressure sealable interlocking rib and groove closure such as of a type exemplified by ZIPLOCK®-type closure 14.

Alternatively, bag containers can be sealed by slider zipper type closures. Alternatively, the bag may be a self seal bag with tape on the access opening or lip or the tissue/collection container 10 can be sealed by a clamp or by fusion of the container material such as by heat. The 3-way connector 100 is connected via tubing 60 to a sample hub, such as for example, needle hub 70 containing a needle. The needled hub 70 can further be connected to a needle protective bell 80. Needle protective bell 80 provides a sterile environment for collection of sample aliquots from the needle into one or more sample tubes or bottles. The protective bell also serves to protect medical personnel from accidental needle sticks. In one embodiment, the collection package is provided in a kit that contains test vials and/or bottles and the needle protective bell is dimensioned to fit and receive the vials and/or bottles.

Suitable tubing sizes can be readily determined by in accordance with capacity to support a sufficient flow rate to carry through the process. For one non-limiting example of tubing dimensioned to provide sufficient flow rate, tubing having an internal diameter of 3 mm, an outside diameter 4 mm, and with a length varying from 10 to 20 cm was found to be suitable in accordance with one embodiment of the invention. Other tubing dimensions can be employed in accordance with flow rate requirements as well as other dimensioning considerations including ergonomic and surgical suite constraints as well as optimization of manufacturing, folding, packing, shipping, storage, and sterilization considerations. The tubing can be clamped off by a clamp such as Roberts clamp 90. Other suitable closures can be alternatively employed such as, for example, roller clamps.

In the depicted embodiment, a further 3-way connector 100 is present on the tubing and further connects via tubing 50 to a second 3-way connector 200. The second 3-way connector 200 is connected via tubing 110 to a further sampling hub such as luer lock 120 having a protective cap 130.

In one embodiment of the invention, a filter apparatus can be attached sampling hub 120 and a volume of sampling fluid can be run through the filter apparatus to collect and concentrate microbial organisms or other particulates in the sampling fluid. The filter disk can then be sampled or cultured by a number of methods that can be readily appreciated by one of skill in the art.

For example, the filtration disk can be removed from the filtration device for culturing by standard techniques. Alternatively, the filtration unit can serve as the culture unit. For an example of such a design, the Sartorius BIOSART 100 Monitors provide a sterile plastic filter funnel in which the lid and base for a Petri dish after media has been added. Alternatively, a filtration unit can be employed that provides a closed system in which the filtration unit serves as the incubation chamber after the sampling fluid has been passed over the filter. An example of a filtration unit configuration where the filtration unit serves as an incubation chamber is commercially available from Sartorius (STERISART Sterility testing).

The 3-way connector 200 is also connected via tubing 140 and tube sleeve 150 to a drain bag 160. In one embodiment, the drain bag 160 can be a blood collection bag generally used in the art. The drain bag is preferably dimensioned to accommodate any portions of the original volume of sample solution from the tissue container that are not otherwise used for testing. The drain bag can be either stored or disposed of a bio-hazardous waste as desired.

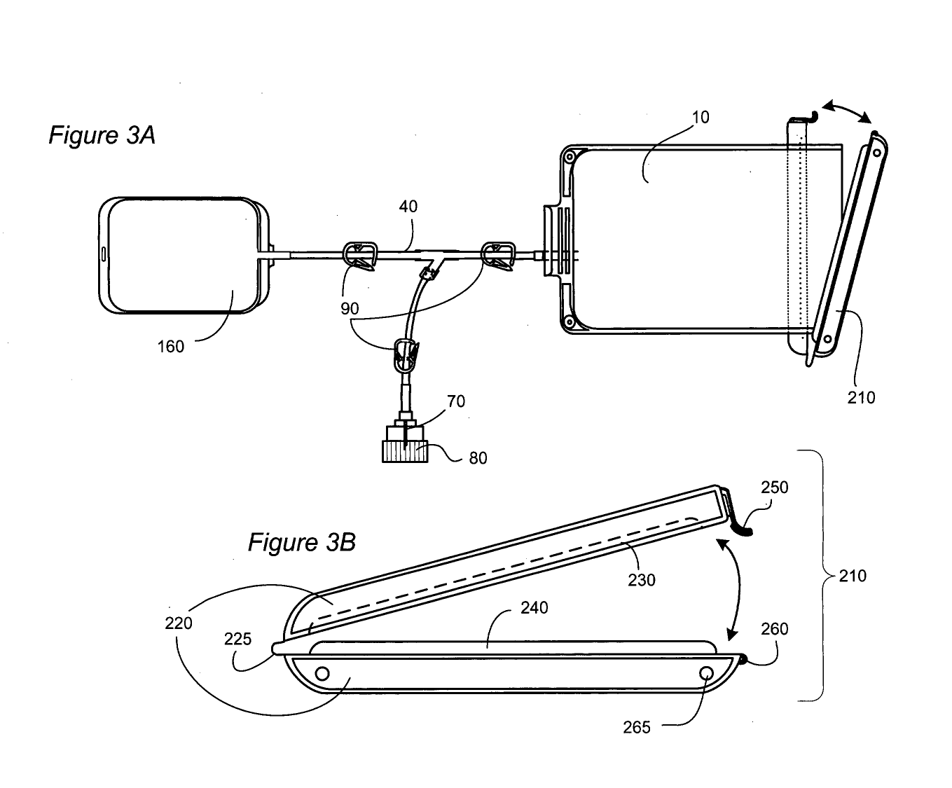

In one embodiment of the invention as depicted in FIG. 3A, a clamp 210 is employed for closure of the tissue/collection container 10. One type of clamp that may optionally be employed has opposing arms 220 connected by a hinge 225. One of the arms 220 has an opposing surface recess channel 230 while the other arm has a raised middle opposing surface ridge 240 that fits into recess/channel 230 of the other arm. The two arms can be held together by an openable clip 250 at the end of one of the arms that locks into place over a prominence 260 at the end of the opposing arm as the arms are closed. The clamp may optionally have one or more hanging hole(s) 265 for hanging and draining the tissue/collection container 10 by gravity when the clamp is closed. The clamp may be manufactured out of many suitable materials such as, for example, plastic that are sufficiently strong to remain sealably closed and may be sterilized.

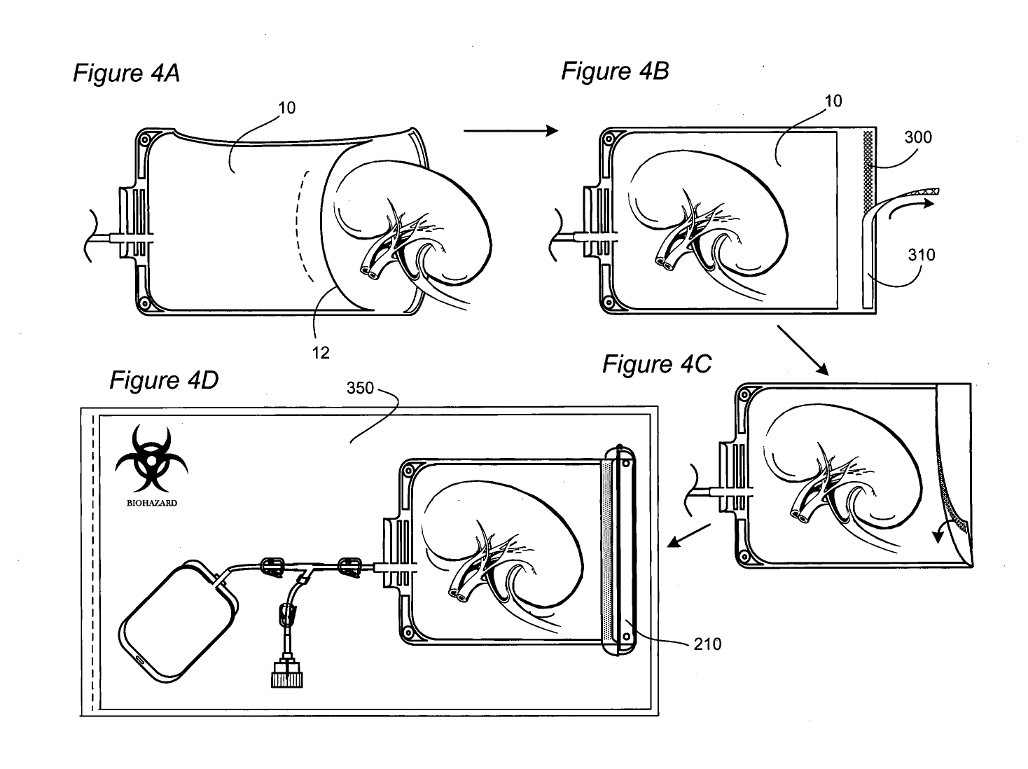

In another embodiment of the invention, and as depicted in FIG. 4B, tissue/collection container 10 may optionally be a bag that has one side wall that is longer that the other. In the embodiment depicted in FIG. 4B, the one of the sides of tissue/collection container 10 is treated to provide an adhesive strip 300 that is protected until use by adhesive strip cover 310. The adhesive strip 300 can be positioned on either a long flap side as depicted on FIG. 4B or, alternatively, on the short side that is covered in part by the flap. The sealing implementation can be of any type that provides for a reopenable closure including for example but without limitation, adhesive sealing strips, interlocking rib and groove closures such as of a type exemplified by ZIPLOCK®-type closures, slider zipper closures, etc.

Thus, and is depicted in FIG. 4A, tissue container 10 is opened at access opening 12 and a tissue or organ placed in the container. Then, after the tissue is placed in the tissue container 10 as in FIG. 4B, adhesive strip cover 310 is removed and as in FIG. 4C, the long end is folded over to engage the adhesive strip 300. The tissue container may be further sealed using clamp 210 or by heat sealing. Finally, the entire procurement set is placed in a further sterile outer bag 350 for transport and storage and to protect against contamination and accidental spillage. The outer bag may be further provided with a document pouch that may be used to keep documents with specimens during transport and storage.

The outer bag can be sealed by any sort of reopenable closure including without limitation, adhesive sealing strips, interlocking rib and groove closures, sliding zipper closures, clamp closures, and folded closures, or any sort of fusing closure such as by heat sealing.

For example, with heat sealing thermal impulse sealers and constant heat bag sealers may be employed and are widely available, including those manufactured to be FDA compliant for medical indications. Such sealers are able to fuse a number of different materials including polyethylene, polystyrene, polypropylene, polyurethane, laminated polylined bags, polyvinyl acetate (PVA), polyvinylidene chloride (i.e. as in SARAN® type wrap), nylon, and other heat sealable materials.

The present invention also provides methods for sterile manipulation of tissues for transplantation using an integral procurement and washing, sampling, and/or storage system. In one embodiment, a sterile collection package, including integral tissue container, tubing set and drain bag according to the invention, is provided to the operating suite for tissue/organ collection. The tissue container can optionally be variously dimensioned to accommodate tissues of different dimensions including bone, ligaments, musculoskeletal tissue, vascular tissue including venous and arterial tissue, skin, dura mater, heart valves, ocular tissue including corneas, and vascularized organs for transplantation.

After the tissue(s)/organ(s) are ready for removal from the donor, the sterile wrapping on the collection package is opened. The access opening of the sterile tissue container is prepared in an open position and the tissues or organs are placed in the container. A fluid is added to the tissue container before or after the tissue is placed in the tissue container. The fluid can be a “washing” solution which can be the same or different from a solution that is subject to microbial sampling and thus might be termed a “sampling solution.” Alternatively, the fluid might be the same or different from a solution that is used for tissue transport or storage. If washing is desired, the tissue container is prefilled with solution, the access opening of the container is sealed after deposition of the tissue, and the tissue is agitated with washing solution, e.g. a saline or physiologically compatible solution. Alternatively the tissue can be washed in a more complex storage or transport solution. If the container is not prefilled, than a solution, such as a washing solution, is added prior to sealing the access port. Alternatively the washing solution can be introduced through the transfer hub. Various volumes of washing solution can be used, depending on the dimensions of the tissue container and the type and/or condition of the tissue or organ. After the tissues are deposited together with solution in the tissue container, and the container has been sealed, the tissue container is agitated or rotated to wash the tissue. The wash step can be repeated as desired and the wash solution ultimately removed from the system. If desired, storage or organ transport solution can then be added to the system. Alternatively, if desired the tissues and organs can be placed immediately in contact with storage or transport solution without prewashing.

As note above, solutions for sampling can be added to the tissue container before or after the tissue is placed in the tissue container. The tissue container can be prefilled with sampling solution, e.g. saline, phosphate buffer or other suitable solution, in which case the access opening of the container is sealed after placement of the tissue therein and the tissue manipulated with the sampling solution. If the container is not prefilled, then sampling solution is added prior to sealing the access port. Alternatively, if the tissue container is prefilled with water or a simple solution, then a concentrated additive solution can be added to provide the particular desired solution of the procurement team, prior to sealing the access port. Various volumes of sampling solution can be used depending on the size of the container, the harvested tissues and the desired number of tests.

In one embodiment where the system is not filled or is not filled to desired capacity during manufacturing, the collection package may optionally include a separate sterile fluid container that includes an appropriate volume of solution that may be used for washing, sampling, and/or as a transfer solution. The volume of such solution may be universal regardless of the volume of the tissue container or may provide a premeasured amount that is suitable for the specific individual volume of the tissue container such that the procurement team simply adds the entire contents of the fluid container to the procurement system, either through the access opening or the transfer hub, and does not have to be concerned with measuring the appropriate amount. For example, for a tissue container that is approximately 9″ (230 mm)×12″ (305 mm), a volume of from approximately 90 ml to approximately 180 ml of sampling solution can be used depending on the volume of the tissue and the volume of fluid need for each test. A drain container having any volume able to accommodate any excess fluid may be employed, such as for example commercially available blood bags or other containers having volumes ranging from approximately 200 to 500 ml. After the tissues are deposited together with solution in the tissue container, and the container has been sealed, the tissues can be mixed with the sampling solution by, for example, rotating the tissue container 10 times.

Aliquots of the sampling solution are then drained via the sampling hub into one or more testing containers for microbial testing. In one embodiment, the sampling hub comprises a needle hub having a needle through which sampling solution is drawn into microbial testing containers. Alternatively, sampling solution can be withdrawn by a syringe through the sampling hub. A number of culture media and culture tubesibottles are generally available in the art for microbial testing. For example, bioMerieux, Inc. (Durham, N.C.) provides a number of culture media and culture bottles to test for aerobic and anaerobic microbes in blood or bodily fluid. These culture tubes/bottles and testing bottles are herein collectively termed “sample assay containers.” Thus, in one embodiment, microbial testing can be performed by using the needle in the needle hub 70 (together with the needle protective bell 80 if necessary) to penetrate the top of a culture bottle containing medium for aerobic microbial culture, and collecting sampling solution (e.g. 10 ml) directly into the culture bottle which is prefilled with the proper amount of culture fluid. The same procedure can be repeated on culture bottles containing media for anaerobic microbial culture. In one embodiment, the collection package includes microbial testing bottles for anaerobic and aerobic testing and includes a needle hub and protective bell that are dimensioned to fit the testing bottles.

After collecting samples of the sampling solution for microbial testing, the remaining sampling solution can be drained into the drain container. Sampling solution in the drain container can be discarded or saved for further testing. For example, the sampling solution in the drain container can be tested for microbial growth by first draining the solution onto a filter, which is then cultured for microbial growth.

Eventually, residual amounts of the sampling solution are drained from the tissue container, leaving only the tissues in the sealed tissue container together with a desired amount of fluid such as for example tissue cold storage or cryopreservation solution. The tissues can then be transported and stored in the tissue container until transplantation pending microbial testing results.

In one embodiment of the invention, a collection package is provided for microbial testing of surgical tools and devices. The collection package provides for packaging and storage of surgical tools that have been used in procedures that may post a risk of infection if reused after conventional sterilization, including in particular surgical instrumentalities that have been used in neurological procedures. In such procedures, there is an increased risk that unconventional transmissible agents, such as prion agents, may be involved in disease pathology. Prion agents are notoriously difficult to inactivate by conventional means such as steam sterilization. Many surgical items are made for multiple-use and are cleaned by the hospital between uses on different patients. In addition, many hospitals are now working with third party reprocessors, who take single use medical devices, clean and sterilize them, and sell them back to the hospital to be used again. Microbial testing using the presently disclosed collection and testing package may provide an avenue for safe sampling of the tools such that they may be cleaned and reused if testing is negative. In the context of testing of surgical instrumentalities, the “tissue container” 10 of FIGS. 1-4 is termed a “collection” container. The instrumentalities are manipulated within the package and sampled for contagious agents, wherein the manipulation is one or more of washing of the instruments in the container, sampling of the wash solution for microbial contamination, and storage of the instruments until test results are received. Test results determine the cleaning and sterilization protocol for the instruments.

In another embodiment of the invention, the collection package is used in forensic applications. As used herein “forensic samples” means tissues and samples, including non-human articles, which may be contaminated with human matter and collected from crime victims and crime scenes. Such forensic samples may be collected and sealed in the collection container of the present invention. In this context, the “tissue container” 10 of FIGS. 1-4 is termed a “collection” container. The washing and sampling systems of the present invention may be employed to provide large volume comprehensive sampling for forensic analysis, including microbial, DNA and toxicology screens. In one method, various fluid samples are removed through one or more collection hubs off the tubing set, while the bulk of the fluid may be saved for later analysis in the drain container 160. Alternatively, filtration of a large volume of fluid through a filter disk allows for concentration of particulates including hairs, fibers, metal fragments, and locality specific indicia including pollens, etc. Provision of labeling surfaces on the collection container, as well as use of an outer bag that is further provided with a document pouch may be used to keep documents with specimens during transport and storage, and allows for robust chain of custody control as is required in forensic applications.

The foregoing description of preferred and other embodiments is not intended to limit or restrict the scope or applicability of the inventive concepts conceived of by the Applicants. In exchange for disclosing the inventive concepts contained herein, the Applicants desire all patent rights afforded by the appended claims. Therefore, it is intended that the appended claims include all modifications and alterations to the full extent that they come within the scope of the following claims or the equivalents thereof.

1. A collection package for microbial or forensic testing, comprising: a collection container dimensioned to accommodate a sample and a volume of fluid, the collection container having a sealable access opening, the sealable access opening extending substantially across one end of the collection container and dimensioned for placing the sample in the collection container;
at least one drain container dimensioned to accommodate at least a portion of the fluid; and
at least one sterile tubing set for providing a controllable fluid path between the collection container and the drain container, wherein the tubing set comprises at least one transfer hub disposed between the collection container and the drain container.

2. The collection package of claim 1, wherein the transfer hub comprises a sample hub.

3. The collection package of claim 2, wherein the sample hub comprises a protective bell for receiving a sample assay container and includes a needle for delivery of an aliquot of the fluid into a sample assay container.

4. The collection package of claim 1, wherein the the sealable access opening is opened and closed by a releasable closure.

5. The collection package of claim 1, wherein the the sealable access opening is adapted for sealing by welding or fusing.

6. The collection package of claim 1, wherein the transfer hub is selected from the group consisting of: a luer lock fitting, a needle penetrable stopper, a septum syringe port, a safety connection device, a valve, a needle hub, and combinations thereof.

7-9. (canceled)

10. A method of tissue procurement and sterile manipulation, comprising:
providing an integral, sterile tissue collection package that comprises a tissue container having sealable access opening, a tubing set and a drain container, wherein the tubing set provides a sterile fluid connection from the tissue container to the drain container;
adding a fluid solution to the tissue container;
collecting a tissue and placing the tissue into the tissue container through the access opening,
manipulating the tissue in the fluid solution; and
storing the tissue in the tissue container.

11. The method of claim 10, wherein the tissue is selected from the group consisting of: bone, ligaments, musculoskeletal tissue, vascular tissue including venous and arterial tissue, skin, dura mater, heart valves, ocular tissue including corneas, vascularized organs for transplantation, diseased tissues, and tissues for forensic testing.

12. The method of claim 10, wherein at least a portion of the fluid solution is added to at least one of the tissue container and the drain container during manufacture.

13. The method of claim 10, wherein at least a portion of the fluid solution is added to tissue container at the time of the procurement procedure.

14. The method of claim 10, wherein the manipulating comprises washing.

15. The method of claim 10, wherein the manipulating comprises sampling.

16. The method of claim 10, wherein the tubing set further comprises a transfer hub that comprises one ore more of the group consisting of: a luer lock fitting, a needle penetrable stopper, a septum syringe port a safety connection device a valve, a needle hub, and pluralities or combinations thereof.

17. (canceled)

18. A method of sample procurement and microbial or forensic testing, comprising:
placing a test article into a sterile collection container through a sealable access opening in the collection container, the sealable access opening extending substantially across one end of the collection container and dimensioned for placing the test article in the collection container, wherein the collection container is connected via a tubing set to a drain container and wherein the tubing set comprises a sampling hub disposed between the collection container and the drain container;
adding a volume of sampling solution to the collection container, either before or after placement of the test article therein;
mixing the article with the sampling solution;
draining an aliquot of the sampling solution via the sampling hub into one or more testing containers for detection of microbes, DNA, or foreign matter;
draining excess sampling solution into the drain container; and
storing the test article in the collection container.

19. The method of claim 18, wherein the test article is selected from the group consisting of: tissues for transplantation, diseased tissue, forensic samples, and surgical instrumentalities.

20. The method of claim 19, wherein the tissues for transplantation are selected from the group consisting of: ligaments, skin, dura mater, heart valves, corneas, and vascularized organs for transplantation.

21. The method of claim 18, wherein the sampling hub comprises a needle for delivery of an aliquot of the sampling solution into the testing container.

22. (canceled)

23. The method of claim 18, wherein the sampling hub comprises one ore more of the group consisting of: a luer lock fitting, a needle penetrable stopper, a septum syringe port, a safety connection device, a valve, a needle hub, and combinations thereof.

24. The method of claim 18, wherein the microbes are selected from the group consisting of: bacteria, mycoplasma, parasites, fungi, viruses, and atypical agents.

25. The method of claim 18, further comprising filtering a portion of the sampling solution over a filter, wherein the filter is then tested for the presence of microbes, DNA, foreign matter, and combinations thereof.
